(19) United States
(12) Patent Application Publication
(10) Pub. No.: US 2007/0042479 A1
(43) Pub. Date: Feb. 22, 2007

(54) Conversion of carbon dioxide to methanol in silica sol-gel matrix

(75) Inventors: Bakul C. Dave, Carbondale, IL (US); Mukti S. Rao, Mundelein, IL (US); Marci C. Burt, Clay City, IL (US)

Correspondence Address: THOMPSON COBURN, LLP, ONE US BANK PLAZA, SUITE 3500, ST LOUIS, MO, 63101, US

(73) Assignee: Southern Illinois University at Carbondale

(21) Appl. No.: 11/207,124

(22) Filed: Aug. 18, 2005

(51) Int. Cl. C12P 7/04

(52) U.S. Cl. 435/157

(57) ABSTRACT

In a sequential enzymatic reduction of carbon dioxide to methanol in which electrons are supplied by conversion of NADH to NAD⁺, NADH may be regenerated from the NAD⁺ by conversion of lactate to pyruvate by lactate dehydrogenase enzymes.

### BACKGROUND OF THE INVENTION

1. Field of the Invention

The present invention relates to chemical reductions catalyzed by dehydrogenase enzymes and more particularly to the implementation of such reductions in the synthesis of methanol.

2. Description of the Related Art

Methanol is used in a wide range of applications. Among such applications may be noted its use in the production of formaldehyde, in automotive anti-freeze, in a variety of chemical syntheses, as a general solvent, as an aviation fuel (for water injection), as a denaturant for ethyl alcohol, and as a dehydrator for natural gas. Conventional techniques for the production of methanol include high-pressure catalytic synthesis from carbon monoxide and hydrogen, partial oxidation of natural gas hydrocarbons, and purification of pyroligneous acid resulting from destructive distillation of wood.

Various techniques for synthesizing methanol from carbon dioxide are also known. As noted in Enzymatic Conversion of Carbon Dioxide to Methanol: Enhanced Methanol Production in Silica Sol-Gel Matrices, J. Amer. Chem. Soc. 1999, 121, 12192-12193 (published on the World Wide Web on Dec. 9, 1999), partial hydrogenation of carbon dioxide has been carried out by means of heterogeneous catalysis, electro-catalysis, and photocatalysis, with oxide-based catalysts being used predominantly for industrial fixation of carbon dioxide. Moreover, the present inventor's U.S. Pat. No. 6,440,711 describes a method of converting carbon dioxide to methanol by a dehydrogenase synthesis scheme that may be carried out in a sol-gel.

Derivation of methanol from carbon dioxide has several obvious advantages. For example, carbon dioxide is plentiful, readily available (indeed, omnipresent), and extremely inexpensive, to say the least. In addition, whereas use of many resources may lead to undesirable depletion of that resource, rising levels of carbon dioxide have been associated with what has been referred to as the “greenhouse” effect, which has been theorized to be a contributing factor to global warming. Thus, moderate removal of carbon dioxide from the atmosphere is viewed as beneficial rather than detrimental.

However, conventional methods for synthesizing methanol from carbon dioxide also suffer from certain drawbacks. Such drawbacks include inefficiencies, costs, high energy consumption, and the need for special equipment adapted for high temperature or highly corrosive environments. For example, one common commercial method of methanol synthesis is by reduction of carbon dioxide in the presence of oxide catalysts. However, this synthesis produces partially reduced species as by-products, thereby not only creating impurities but also resulting in limited conversion efficiency. Moreover, the process is carried out at high temperatures, requiring special equipment for accommodating and maintaining such temperatures as well as high energy input.

Various other procedures for reduction of carbon dioxide by enzyme-catalyzed reactions also have been described, but such processes either have not been directed to methanol production or involve various drawbacks. Thus, for example, in CO₂ Reduction to Formate by NADH Catalysed by Formate Dehydrogenase from Pseudomonas oxalaticus, Ruschig et al., Eur. J. Biochem. 70, 325-330 (1976), a direct reduction of carbon dioxide by formate dehydrogenase using substrate amounts of NADH is disclosed. The carbon dioxide is reported to have been reduced to formate via carbonate formation in a reaction requiring strict anaerobic conditions to prevent oxygen-induced oxidation of the NADH.

Parkinson and Weaver also describe the production of formate via the formate dehydrogenase catalyzed reduction of carbon dioxide. Photoelectrochemical Pumping of Enzymatic CO₂ Reduction, Nature 309, 148 (1984). In their process, Parkinson and Weaver report that a 150 watt tungsten/halogen lamp generated electrons from the semiconductor indium phosphide to reduce methyl viologen (MV²⁺), which they state mediated the enzyme linked reduction of carbon dioxide to formate. Parkinson and Weaver state that an electrochemical reaction was used to reduce MV²⁺.

Mandler and Willner discuss relaying photoinduced electrons generated by the (Ru(bpy)₃)²⁺/MV²⁺ system to an electron transfer molecule such as 2-mercaptoethanol or cystine. Photochemical Fixation of Carbon Dioxide: Enzymic Photosynthesis of Malic, Aspartic, Isocitric, and Formic Acids in Artificial Media, J. Chemical Soc., Perkin Trans., 997 (1988). According to Mandler and Willner, the 2-mercaptoethanol so energized reduced NADP⁺ to generate NADPH, which mediated the enzyme-induced carboxylation of pyruvate to malate. Likewise, Mandler and Willner show that cysteine is capable of donating electrons in the formate dehydrogenase-induced reaction of CO₂ to formate. Mandler and Willner note that the formate dehydrogenase activity is problematic because it decays rapidly upon exposure to light, and postulate that since the decarboxylation of formic acid is so energetically favorable, NADH is too weak a reducer to enable efficient production of formate.

Kuwabata et al. describe the sequential reduction of carbon dioxide to methanol by use of formate dehydrogenase and methanol dehydrogenase enzymes, wherein electrons are generated electrochemically and either pyrroloquinolinequinone (PQQ) or MV²⁺ is used as the electron carrier. Thus, the Kuwabata et al. technique is an electrolytic process that requires everything essential to such processes, including an elecrolytic bath, electrodes, and electrical current input, and also requires use of PPQ or MV²⁺. Moreover, Kuwabata et al. report that the electrolysis had to be carried out in the dark to maintain the durability of the formate dehydrogenase enzyme.

The method of U.S. Pat. No. 6,440,711 addressed the deficiencies of these various processes by contacting a combination of dehydrogenase enzymes with carbon dioxide, effecting a low temperature, high-yield reduction of the carbon dioxide to methanol that was highly selective, resulting in high yield of methanol and little if any formation of undesirable by-products without the need for special equipment designed for high temperatures or corrosive environments. Moreover, it was found that by entrapping the enzymes within a matrix of micropores (i.e., very small pores)—especially, nano-pores on the order of billionths of meters in diameter—and particularly when driven by the presence of an abundance of donative electrons, for instance from an excess of the reduced form of a cofactor of the enzymes (such as nicotinamide adenine dinucleotide (NADH)) relative to the unreduced form (such as nicotinamide adenine dinucleotide (NAD⁺)), the equilibrium of the reaction can be shifted so that the tendency toward oxidation can be reversed, causing the reaction to proceed as a reduction of carbon dioxide to methanol. However, that method employs photosystem II for regeneration, which has been found to be difficult to isolate and to stabilize.

Accordingly, a new technique for synthesis of methanol, and especially conversion of carbon dioxide to methanol, that alleviates such drawbacks is desired. In particular, a low temperature, highly efficient technique for production of methanol from carbon dioxide is desired.

### SUMMARY OF THE INVENTION

Briefly, therefore, the present invention is directed to a novel method for serial reduction of the carbon dioxide to methanol by formate dehydrogenase enzymes, formaldehyde dehydrogenase enzymes and alcohol dehydrogenase enzymes in the presence of reduced nicotinamide adenine dinucleotide as a terminal electron donor wherein the serial reduction comprises a series of reduction reactions to which a terminal electron is donated by oxidation of the reduced nicotinamide adenine dinucleotide to nicotinamide adenine dinucleotide and wherein the nicotinamide adenine dinucleotide is regenerated back to reduced nicotinamide adenine dinucleotide by lactic dehydrogenase enzymes.

The present invention is also directed to a novel method for conversion of carbon dioxide to methanol comprising introduction of carbon dioxide and lactate to a substrate containing formate dehydrogenase enzymes, formaldehyde dehydrogenase enzymes, alcohol dehydrogenase enzymes, lactate dehydrogenase enzymes and reduced nicotinamide adenine dinucleotide thereby to produce methanol and pyruvate.

The present method is further directed to a novel composition comprising a sol-gel matrix containing formate dehydrogenase enzymes, formaldehyde dehydrogenase enzymes, alcohol dehydrogenase enzymes, lactate dehydrogenase enzymes and reduced nicotinamide adenine dinucleotide.

Among the several advantages found to be achieved by the present invention, therefore, may be noted the provision of a method for synthesis of methanol from carbon dioxide at low temperature; the provision of such method that is energy efficient; the provision of such method that yields high conversion rates; the provision of such method that avoids the need for special equipment adapted to high temperature or highly corrosive environments; the provision of such method that avoids the drawbacks associated with the use of photosystem II for regeneration of NADH; and the provision of a composition that can be employed in such methods to achieve the noted advantages.

### BRIEF DESCRIPTION OF THE DRAWINGS

FIG. 1 is a schematic illustration of the series of reduction reactions of this invention; and

FIG. 2 is a schematic illustration of the reduction of this invention from carbon dioxide to methanol, with regeneration and recycling of NADH.

### DETAILED DESCRIPTION OF THE PREFERRED EMBODIMENTS

In accordance with the present invention, it has been discovered that if, in a sequential enzymatic reduction of carbon dioxide to methanol wherein electrons are supplied by conversion of reduced nicotinamide adenine dinucleotide (NADH) to the oxidized form of nicotinamide adenine dinucleotide (NAD⁺), the NADH is regenerated from the NAD⁺ by lactate dehydrogenase enzymes as opposed to the photosystem II of U.S. Pat. No. 6,440,711, not only does the conversion to methanol have the benefits afforded by the process of U.S. Pat. No. 6,440,711, but the drawbacks associated with photosystem II—that is, the instability of photosystem II and the difficulty of isolating it—are avoided. Moreover, it has been found that this technique produces, as a by-product, pyruvate, which is valuable for a variety of uses.

In short, therefore, the present technique follows the same reaction scheme as disclosed in U.S. Pat. No. 6,440,711, except that the regeneration of NADH from NAD⁺ is effected by conversion of lactate to pyruvate by lactate dehydrogenase enzymes (LDH). Thus, according to the present invention, the conversion of carbon dioxide to methanol comprises serial reduction of the carbon dioxide to methanol by converting carbon dioxide to formic acid (HCOOH) (or formate ion HCOO⁻) by means of formate dehydrogenase enzymes (Fₐₜₑ DH), converting the formic acid to formaldehyde (HCHO) by means of formaldehyde dehydrogenase enzymes (F_{ald} DH), and converting the formaldehyde to methanol by means of alcohol dehydrogenase (ADH) enzymes, with electrons and hydrogen ions provided in each of these steps by the conversion of NADH to NAD⁺, with regeneration of the NADH from the NAD⁺ effected by conversion of lactate to pyruvate by means of LDH. Physically, this process may be carried out by contacting carbon dioxide with Fₐₜₑ DH to form formic acid, contacting the formic acid with F_{ald} DH to form formaldehyde, and contacting the formaldehyde with ADH to form methanol, with each of these steps carried out in the presence of NADH, LDH and lactate.

Preferably, the process is continuous, with input, preferably continuous input, of carbon dioxide and lactate, with continuous output of methanol and pyruvate. This may be carried out by contacting carbon dioxide and lactate with a substrate containing formate dehydrogenase enzymes, formaldehyde dehydrogenase enzymes, alcohol dehydrogenase enzymes, lactate dehydrogenase enzymes and reduced nicotinamide adenine dinucleotide, such as by continuously introducing the carbon dioxide and lactate to the substrate, while continuously removing methanol and pyruvate. Preferably the substrate is a matrix of micropores (i.e., very small pores)—especially, nano-pores on the order of billionths of meters in diameter—and, the enzymes are entrapped within the matrix.

As was found with the NADH regeneration afforded by photosystem II in the process of U.S. Pat. No. 6,440,711, it also has been found that lactate and LDH allows repeated regeneration, recycling and re-use of NADH. In other words, after the reduced form of the cofactor (that is, NADH) is oxidized by donating an electron to the dehydrogenase catalyzed reduction scheme of carbon dioxide to methanol, the reduced form of the cofactor may be regenerated from the form resulting from the oxidation (NAD⁺), thereby allowing repeated re-use of a single dose of the cofactor. In fact, it has been found that the reduced cofactor serving as the electron donor can be regenerated and reused not just a few times, but many times. As a result, the method of this invention provides a surprisingly practical, low-cost, low energy consumption and efficient mechanism for converting carbon dioxide to methanol.

Thus, according to the process of the present invention, a matrix containing a certain combination of dehydrogenase enzymes may be contacted with carbon dioxide to induce an ordered series of reduction reactions catalyzed by those enzymes ultimately to produce methanol. In practice, atmospheric carbon dioxide from any source, even atmospheric carbon dioxide may be simply bubbled through water containing a porous matrix that contains the enzymes entrapped in its pores. The NADH (and NAD⁺) and lactate may be present in the water or matrix or both, with lactate preferably continuously replenished by input flow of it to the water and/or matrix and pyruvate preferably continuously removed. Methanol removal may be accomplished, for example, by distillation. Pyruvate may be removed, for example, by precipitation or crystallization. Each reduction step in the reaction scheme from carbon dioxide to methanol also consumes two hydrogen ions (protons) provided by the conversion of NADH to NAD⁺.

### The Matrix

The matrix within which the enzymes are entrapped is, as noted above, a microporous, even nano-porous, structure capable of retaining the enzymes within the pores or interstices, but such that the enzymes also may be exposed to carbon dioxide transported (such as by bubbling) to or through the matrix. The matrix may be in the form of small particles or a powder (perhaps as the result of grinding) and suspended in the medium (e.g., water) in which the series of reduction reactions takes place. As will be explained below in the section discussing regeneration of the terminal electron donor, a particularly advantageous technique for such regeneration involves enzymatic-regeneration.

The matrix may be made up of an inorganic solid such as an oxide, zeolite, meso-porous silicates, extended networks or layered materials. However, it has been found that sol-gel glasses are especially well suited to the subject process and so are particularly desirable matrix materials. The sol-gel process is a well-known technique involving the transition of a solution system from a liquid “sol” into a solid “gel” phase. Sols usually are prepared from a precursor such as an inorganic metal salt, a metal alkoxide or another metal organic compound. Preferably, the sol-gel glass useful in the subject invention is based on a silica precursor, such as those of the type (OR)₄ Si, RSi(OR)₃, RSi(OR)₂, or (OR)₃Si-spacer-Si(OR)₃, wherein R is an alkyl, alkenyl, alkynyl, or aryl group, and the spacer unit comprises an organic unit, an inorganic unit, or a combination thereof. Although alkoxides or silicon are preferred, other metal oxides, such as those prepared by adding methanol, ethanol, isopropanol or other similar alcohols to the oxides of metals or non-metals such as aluminum, titanium, zirconium, niobium, hafnium, chromium, vanadium, tungsten, molybdenum, iron, tin, phosphorus, sodium, calcium, and boron, or combinations thereof, are candidates for precursors of the sol-gels of this invention. Nevertheless, tetramethylorthosilicate (TMOS) has been found to be a particularly useful precursor, and tetraethylorthosilicate (TEOS) and other active silicon compounds are preferred as well.

The precursor is subjected to a series of hydrolysis and polymerization reactions to form the “sol”—a colloidal suspension. Thin films can be deposited on a substrate such as by spin-coating or dip-coating, if so desired. Upon casting the “sol” in a mold, a “wet gel” is formed. The wet gel can be dried and heated until a dense material forms. However, if the liquid in a wet gel is extracted under a supercritical condition, a highly porous and extremely low-density material called an “aerogel” is formed. The resulting porous material is referred to as a “sol-gel glass.” The average pore diameter in sol-gel glass typically ranges from 2 nm to 200 nm. The pores are interconnected and may be doped with almost any gas, liquid or solid.

### The Enzymes

A combination of formate dehydrogenase (Fₐₜₑ DH), formaldehyde dehydrogenase (F_{ald} DH), and alcohol dehydrogenase (ADH) enzymes has been found to be an especially effective combination of enzymes for catalyzing the ordered series of reductions, although it is believed that methanol dehydrogenase enzymes may be substituted for the alcohol dehydrogenase enzymes. The series of reduction reactions catalyzed by the enzymes has been found to proceed as follows. First, the formate dehydrogenase enzyme (e.g., E.C. 1.2.1.2, E.C. 1.2.1.43, or E.C. 1.2.2.3) induces a formate-catalyzed reduction of the carbon dioxide to formate. The formaldehyde dehydrogenase enzyme (e.g., E.C. 1.2.1.46) then catalyzes the reduction of the formate to formaldehyde. And the alcohol dehydrogenase enzyme (e.g., E.C. 1.1.1.1, E.C. 1.1.1.2, E.C. 1.1.1.71, or E.C. 1.1.99.8) then catalyzes the reduction of the formaldehyde to methanol. Of course, variations of these enzymes, and even mutant variations, that maintain the described catalytic functionality may be employed in place of any or all of these enzymes. It is therefore contemplated that such site-specific variants may be employed in place of the preferred enzymes without departing from the scope of this invention and discussion herein of the noted dehydrogenase enzymes is intended to encompass such variants as well. The matrix also contains lactate dehydrogenase enzymes (LDH) for regeneration of the NADH from NAD⁺.

### Incorporation of the Enzymes in the Matrix

In the subject invention, the matrix is doped with the combination of enzymes discussed above. U.S. Pat. No. 5,200,334 to Dunn et al., incorporated herein by reference, describes a sol-gel process for the preparation of porous glass structures having active biological material such as protein entrapped therein, and notes that encapsulated or entrapped enzymes are used a micro-catalysts. Although the patent nowhere discloses or suggests the use of dehydrogenase enzymes, or particularly those dehydrogenase enzymes identified above, according to the present invention, the enzymes employed in this invention may be encapsulated into a sol-gel glass by the procedure described in U.S. Pat. No. 5,200,334. It has been found that use of enzymes trapped in a sol-gel matrix is three-to-four times more efficient at converting carbon dioxide to methanol than is the use of a mere solution of the enzymes.

### Terminal Electron Hydrogen Ion Donation by Nicotinamide Adenine Dinucleotide

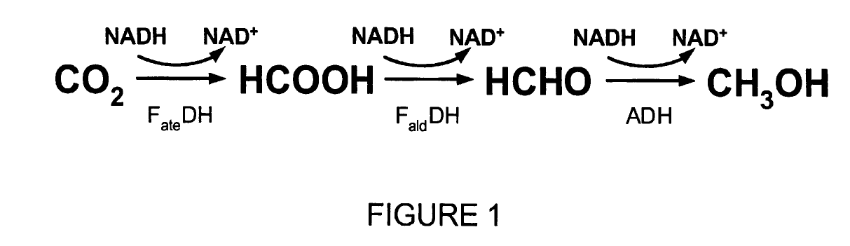

As can be seen from the reaction scheme illustrated in FIG. 1, each of the reduction steps in the reaction scheme from carbon dioxide to methanol consumes an electron, which must be donated from some source. However, if a free electron is provided, it may be introduced at any of a number of sites on the enzymes, resulting in undesirable side reactions. Therefore, for the aforementioned reactions to proceed as described, a mechanism to deliver the electron to the appropriate sites on the enzymes is needed. It is preferred that such a mechanism for donating electrons, such as a carrier that delivers the electron to the enzymes site-specifically, be included with the matrix (or water) as well. Cofactors of the enzymes provide such site-specific delivery and so are the preferred terminal electron donors. It has been found that reduced nicotinamide adenine dinucleotide (NADH) is especially well-suited to act as a terminal electron donor for each of the three reduction reactions. Thus, the overall synthesis may be shown schematically as in FIG. 1.

The conventional direction of the series of reduction reactions carried out in this invention is actually the reverse of that carried out herein; that is, conventionally, the reactions tend to oxidation, which would result formation of carbon dioxide from methanol. However, it has been found that the thermodynamics of the reactions can be shifted so that the reverse reactions (that is, the reductions) are favored if the reduced cofactor is present in great excess of that called for by the stoichiometry of the oxidation reactions. As can be seen from FIG. 1 for the case of NADH, the stoichiometry calls for three moles of NADH for conversion of one mole of carbon dioxide to one mole of methanol. Thus, where the electron donor is NADH, for example, the reactions herein should be carried out in the presence of about 3,000 moles of NADH per mole carbon dioxide converted. Viewing it differently, it can be seen also from FIG. 1, that the reactions scheme yields three moles of NAD⁺ per mole of carbon dioxide converted, and so the ratio of NADH to NAD⁺ should be maintained on the order of 1,000 or more.

### The Hydrogen Ion Donor

Although each reduction reaction in the overall scheme of this invention consumes two hydrogen ions, it is not necessary that the method include addition of a separate hydrogen ion donor for that sole and specific purpose. The hydrogen ions consumed in each of the reduction reactions may be derived from another additive (such as an acid), or from some other mechanism for supplying the ions but, alternatively, they may be derived from the water itself if the process of the subject invention is carried out in an aqueous system, or from the terminal electron donor. If they are derived from the water, it may be preferred that the extraction of the hydrogen ion be accompanied by some other process to generate the ions or to compensate for the ramifications of the extraction. In other words, the continual removal of hydrogen ions from the water might increase the pH thereof, requiring—at least at some point—adjustment downward of the pH, such as by the addition or incorporation of a buffer or acid. Generation of the hydrogen ions from the water may be carried out, for example, electrochemically, by addition of hydrogen gas, or by conversion of the water to oxygen gas.

### Regeneration of the Terminal Electron Donor and/or Hydrogen Ion Donor

As noted above, NADH has been found to be an excellent terminal electron donor and hydrogen ion donor in the process of this invention. However, NADH is relatively expensive. Therefore, it is desirable that if NADH is used, that the NADH be regenerated so that it can be recycled for repeated use. Surprisingly, it has been found that not only can the NADH indeed be regenerated from the NAD⁺, but that it can be regenerated simply and “automatically” without a need for any additional steps that would involve, for example, removal of the NAD⁺ from the reaction vessel, extra treatment and return to the reaction vessel and without any significant interference with the reduction reactions of this invention. In particular, it has been discovered that by simply adding lactate dehydrogenase enzymes (LDH) to the reaction vessel—or to the matrix itself—and carrying out the reactions in the presence of lactate, the NADH can be regenerated and recycled automatically and continuously.

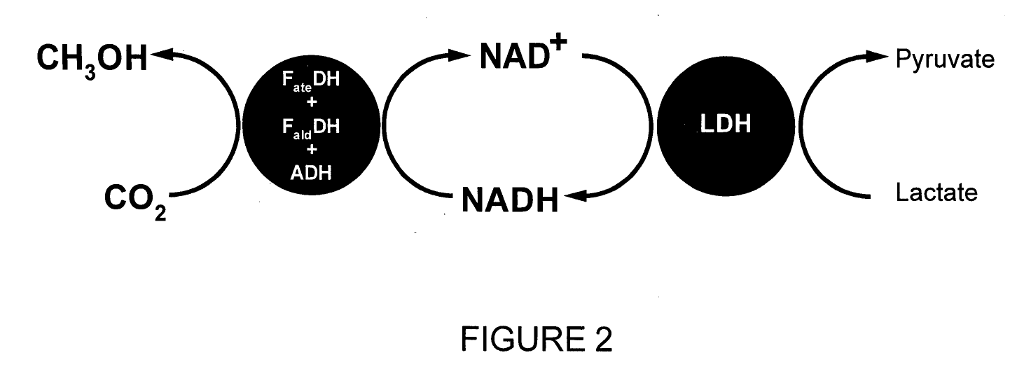

Thus, upon incorporation of the LDH into the reduction system of this invention and exposure of the LDH to lactate, the LDH converts lactate to pyruvate and provides electrons to the NAD⁺ to produce NADH. See FIG. 2 for a schematic representation. According to the overall reaction, therefore, carbon dioxide, water and lactate are converted to methanol and pyruvate. The reaction scheme has been found to run—apparently—indefinitely on the initial doses of the enzyme-containing matrix and NADH, requiring only carbon dioxide, water and lactate as input flows thereafter, and producing methanol and lactate. The LDH may be incorporated into the reaction scheme of this invention by the same techniques discussed above with the other enzymes.

### Contacting the Enzymne-Containing Matrix with Carbon Dioxide

In a preferred embodiment, a sol-gel matrix is prepared as described in Enzymatic Conversion of Carbon Dioxide to Methanol: Enhanced Methanol Production in Silica Sol-Gel Matrices, R. Obert and B. Dave (the inventor herein), J. Am. Chem. Soc. 1999, 121, 12192-12193. In summary, the gel may be prepared by mixing tetramethoxysilane precursor, water and HCl to form a mixture that is then sonicated to form a sol. The sol is added to a stock of the combination of enzymes described above in buffer (pH 7). Where NADH is used as the terminal electron and hydrogen donor, the resulting gel is allowed to age and immersed in a solution of NADH to allow the NADH molecules to diffuse into the gel. The resulting gel containing the four enzymes of this invention and the NADH may be crushed to a particulate or powder form and maintained in suspension in water held in a reaction vessel such as a CSTR.

Carbon dioxide, such as atmospheric carbon dioxide, may be bubbled under constant positive pressure through the water and so diffused through the matrix and contacted with the enzymes entrapped in the matrix. Remaining carbon dioxide and resulting methanol then diffuse out of the matrix and through the water for collection by distillation and the recirculation of the aqueous fraction, if so desired. Alternatively, solid form of carbon dioxide may be employed, for example, by adding it to the matrix-containing aqueous mixture. Lactate may be added and methanol and pyruvate removed as noted above.

The following examples describe preferred embodiments of the invention. Other embodiments with the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1

Two enzyme stock solutions, one containing the enzymes FDH, F_{ald}DH, and ADH, and the other containing the enzymes FDH, F_{ald}DH, ADH, and LDH, were prepared. Each stock solution was prepared by dissolving the noted enzymes in a pH 7 phosphate buffer solution such that the concentration of each enzyme was about 10 mg/mL. TMOS sol was prepared by sonicating a mixture of the precursor TMOS (1.5 mL), water (0.4 mL) and 0.04 M HCl (0.022 mL) for about twenty minutes. Two samples of TMOS sol-gel were prepared, one by mixing a portion of the TMOS sol in a 1:1 volumetric ratio with the first stock solution, the other by mixing a portion of the TMOS sol in a 1:1 volumetric ratio with the second stock solution.

Samples of each of the enzyme stock solutions were mixed with an aqueous solution of NADH (75 mg/mL) in a 1:1 volumetric ratio to produce a total volume in each case of about one milliliter. The mixtures were then exposed to various concentrations (from 0.0066 to 0.264 moles) of solid carbon dioxide for three hours. In some cases, however, gaseous carbon dioxide was bubbled through the mixture while measurements were being made.

In one set of experiments, about a half a gram of the powdered sol-gel samples containing the encapsulated enzymes (with and without LDH) were powdered and kept in contact with pH 7 phosphate buffer (2 mL) to which the solid carbon dioxide was added. An aliquot from the outside solution was withdrawn after three hours for NADH and methanol detection as described below.

In a second set of experiments, about a half a gram of the powdered sol-gel samples containing the encapsulated enzymes and various concentrations of NADH were also kept in solution with pH 7 phosphate buffer (2 mL) and gaseous carbon dioxide was bubbled into the solution for three hours, after which an aliquot from the outside solution was withdrawn and then tested for methanol.

### EXAMPLE 2

To study the feasibility of creating a self-sustaining system to regenerate NADH, the rate of NADH consumption in the first step of the conversion (carbon dioxide to formate ion) by means of FDH was determined. Two solutions containing FDH and NADH (one containing 0.01 M NADH and the other containing 0.025 M NADH) was bubbled with gaseous carbon dioxide. The decrease in NADH was measured using a fluorometer and a UV-visible spectrometer. When the initial NADH concentration was 0.01 M, the emission intensity of NADH at 457 nm decreased over time, from an initial level of about 850 a.u. (arbitrary units) to about 780 after five minutes, to about 550 after about fifteen minutes, to about 400 after about twenty-five minutes, to about 270 after about thirty-five minutes. However, for an initial NADH concentration of 0.025 M, an initial decrease in the emission intensity at 457 nm of NADH from 46 to 37 after five minutes was followed by an increase to about 39.5 after ten minutes, to about 40.5 after twenty minutes, to about 42.5 after about thirty-five minutes, and about 42 after about fifty minutes, indicating that initially the NADH was consumed by the FDH to convert carbon dioxide to methanol.

### EXAMPLE 3

Gaseous carbon dioxide was bubbled into a solution containing FDH, LDH and lactate similarly to the procedures of Example 2, above. NADH generation was measured with a UV-visible spectrophotometer for a fixed lactate concentration of 0.01 M. In this case, the absorbance at 330 nm showed a progressively increasing absorbance over time from an initial reading of about 0.32 to about 0.48 after sixty minutes.

### EXAMPLE 4

NADH emission was measured in a system consisting of powdered gel containing FDH and NADH for various concentrations of solid carbon dioxide. The NADH emission from the outside solution containing solid carbon dioxide was found to increase with increasing moles of carbon dioxide. The luminescence intensity difference at 457 nm was found to be about ten for 0.005 moles of carbon dioxide, about 25 for about 0.013 moles of carbon dioxide, about sixty for about 0.020 moles of carbon dioxide, and about ninety for about 0.025 moles of carbon dioxide.

### EXAMPLE 5

A control experiment was carried out by exposing a TMOS sol-gel containing encapsulated NADH to solid carbon dioxide. The experiment revealed no NADH emission with respect to time, suggesting that increased NADH emission as not due to leaching of NADH from the TMOS sol-gel and that the enzyme FDH is required for the reaction between NADH and carbon dioxide. The rate of decrease in NADH emission was found to increase with amounts of carbon dioxide, from about 0.03 for 0.005 moles of carbon dioxide, to about 0.04 for about 0.013 moles of carbon dioxide, to about 0.08 for about 0.020 moles of carbon dioxide, and to about 0.12 for about 0.025 moles of carbon dioxide, suggesting that with more carbon dioxide present in the solution, more NADH comes out from the sol-gel into the solution to react with carbon dioxide.

NADH formation was measured using LDH and NAD⁺ with respect to the lactate concentration using fluorescence spectroscopy. An initial increase in NADH emission intensity was detected at 457 nm from about 250 for 0.01 M lactate to about 630 for 0.025 M lactate, but the NADH emission intensity decreased to about 100 for about 0.05 M lactate and about 90 for about 0.075 M lactate.

### EXAMPLE 6

A series of experiments was carried out to couple the LDH system with the (FDH+F_{ald}DH+ADH) system to create a self-sustained system for methanol production in accordance with the present invention. A GC column with organic polymer containing cellulose groups and a themal conductivity detector was prepared to detect and quantify the production of methanol in the presence of water. After the column was heated in an oven and held at 125° C. and equilibrated with nitrogen for four hours. The same column was used for all experiments and was run under the following conditions: oven temperature=initial oven temperature=80° C., final oven temperature=245° C., detector temperature=100° C., run time=three minutes.

The first set of experiments was carried out in two types of TMOS sol-gel, one containing FDH, F_{ald}DH and ADH, the other also containing 10 mg/L LDH. Neither type of sol-gel was washed with water or phosphate buffer to remove generated methanol. The amount of methanol produced was found to be greater with the LDH present, indicating that the inclusion of LDH and lactate created a self-sustaining system that produced more methanol. Specifically, the system that included LDH produced in excess of 200 moles of methanol at each level of carbon dioxide employed (ten, fifteen and thirty moles); the system without LDH produced about 173 moles of methanol at ten moles of carbon dioxide, about 185 moles of methanol at fifteen moles of carbon dioxide, and less than about 160 moles of methanol at thirty moles of carbon dioxide. A GC column was run under isothermal conditions, with the initial temperature equal and final oven temperature equal to 105° C., the detector temperature equal to 200° C. and a run time of five minutes. A peak due to the formation of methanol was observed in the system without LDH.

The second set of experiments investigated whether increasing NADH concentration increases carbon dioxide production in TMOS sol-gel with all noted enzymes present. The amount of NADH was varied for each successive gel and the gel was maintained in a 2 mL solution of pH 7 phosphate buffer. Gaseous carbon dioxide was bubbled into the solution for three hours. The GC column was run under isothermal conditions as described in the preceding paragraph. It was found that 0.05 moles of NADH resulted in the production of about 0.2 moles of methanol, 0.21 moles of NADH resulted in the production of about 0.8 moles of methanol, 0.31 moles of NADH resulted in the production of about 1.4 moles of methanol, and 0.42 moles of NADH resulted in the production of about 4.4 moles of methanol.

The third set of experiments was carried out in the manner of the second set, but without LDH. About two thousand times as much of methanol was produced compared to the second set of experiments: 0.05 moles of NADH resulted in the production of about 0.1 mmoles of methanol, 0.21 moles of NADH resulted in the production of about 0.4 mmoles of methanol, 0.31 moles of NADH resulted in the production of about 0.7 mmoles of methanol, and 0.42 moles of NADH resulted in the production of about 2.25 mmoles of methanol.

All references, including without limitation all papers, publications, presentations, texts, reports, manuscripts, brochures, internet postings, journal articles, periodicals, and the like, cited in this specification are hereby incorporated by reference. The discussion of the references herein is intended merely to summarize the assertions made by their authors and no admission is made that any reference constitutes prior art. The inventors reserve the right to challenge the accuracy and pertinence of the cited references.

In view of the above, it will be seen that the several advantages of the invention are achieved and other advantageous results obtained.

As various changes could be made in the above methods and compositions without departing from the scope of the invention, it is intended that all matter contained in the above description as shown in the accompanying drawings shall be interpreted as illustrative and not in a limiting sense.

What is claimed is:

1. A method for conversion of carbon dioxide to methanol comprising serial reduction of the carbon dioxide to methanol by formate dehydrogenase enzymes, formaldehyde dehydrogenase enzymes and alcohol dehydrogenase enzymes in the presence of reduced nicotinamide adenine dinucleotide as a terminal electron donor wherein the serial reduction comprises a series of reduction reactions to which a terminal electron is donated by oxidation of the reduced nicotinamide adenine dinucleotide to nicotinamide adenine dinucleotide and wherein the nicotinamide adenine dinucleotide is regenerated back to reduced nicotinamide adenine dinucleotide by lactic dehydrogenase enzymes.

2. A method as set forth in claim 1 wherein the alcohol dehydrogenase enzymes are methanol dehydrogenase enzymes.

3. A method as set forth in claim 1 wherein the alcohol dehydrogenase enzymes are other than methanol dehydrogenase enzymes.

4. A method as set forth in claim 1 wherein the formate dehydrogenase, formaldehyde dehydrogenase, alcohol dehydrogenase and lactic dehydrogenase enzymes are embedded in a microporous matrix.

5. A method as set forth in claim 4 wherein the microporous matrix is a sol-gel matrix.

6. A method as set forth in claim 1 wherein the regeneration takes place in the presence of lactate.

7. A method as set forth in claim 5 wherein the regeneration takes place in the presence of lactate.

8. A method as set forth in claim 6 wherein the regeneration converts the lactate to pyruvate.

9. A method as set forth in claim 7 wherein the regeneration converts the lactate to pyruvate.

10. A method as set forth in claim 8 wherein the reduction of the carbon dioxide to methanol takes place in water.

11. A method as set forth in claim 9 wherein the reduction of the carbon dioxide to methanol takes place in water.

12. A method as set forth in claim 1 wherein the method is a continuous flow process.

13. A method for conversion of carbon dioxide to methanol comprising introduction of carbon dioxide and lactate to a substrate containing formate dehydrogenase enzymes, formaldehyde dehydrogenase enzymes, alcohol dehydrogenase enzymes, lactate dehydrogenase enzymes and reduced nicotinamide adenine dinucleotide thereby to produce methanol and pyruvate.

14. A method as set forth in claim 13 wherein the substrate is a sol-gel matrix.

15. A method as set forth in claim 14 wherein the method is a continuous process in which nicotinamide adenine dinucleotide is continuously formed from the reduced nicotinamide adenine dinucleotide and the nicotinamide adenine dinucleotide so-formed is continuously regenerated back to reduced nicotinamide adenine dinucleotide.

16. A method as set forth in claim 15 wherein regeneration of reduced nicotinamide adenine dinucleotide from the nicotinamide adenine dinucleotide is catalyzed by the lactate dehydrogenase enzymes with concomitant conversion of lactate to pyruvate.

17. A composition comprising a sol-gel matrix containing formate dehydrogenase enzymes, formaldehyde dehydrogenase enzymes, alcohol dehydrogenase enzymes, lactate dehydrogenase enzymes and reduced nicotinamide adenine dinucleotide.

18. A composition as set forth in claim 17 wherein the sol-gel matrix further comprises nicotinamide adenine dinucleotide.

19. A method for reduction of formaldehyde to methanol by alcohol dehydrogenase catalysis, comprising exposing the formaldehyde to a microporous matrix containing alcohol dehydrogenase enzymes, lactate dehydrogenase enzymes and reduced nicotinamide adenine dinucleotide.

20. A method as set forth in claim 19 wherein the formaldehyde is produced by reduction of formate to the formaldehyde by formaldehyde dehydrogenase catalysis, comprising exposing formaldehyde dehydrogenase enzymes retained in a microporous matrix to the formate.

21. A method as set forth in claim 20 wherein the formate is produced by reduction of carbon dioxide to the formate by formate dehydrogenase catalysis, comprising exposing formate dehydrogenase enzymes retained in a microporous matrix to the carbon dioxide.

22. A method as set forth in claim 19 wherein reduced nicotinamide adenine dinucleotide acts as a terminal electron donor in the reduction.

23. A method as set forth in claim 20 wherein reduced nicotinamide adenine dinucleotide acts as a terminal electron donor in each of the reductions.

24. A method as set forth in claim 21 wherein reduced nicotinamide adenine dinucleotide acts as a terminal electron donor in each of the reductions.

25. A method as set forth in claim 19 wherein the microporous matrix is a sol-gel.

26. A method as set forth in claim 24 wherein the reduced nicotinamide adenine dinucleotide is oxidized to nicotinamide adenine dinucleotide upon donation of terminal electron to the reductions and is regenerated back to reduced nicotinamide adenine dinucleotide by lactate dehydrogenase catalysis to serve again as a terminal electron donor for further reduction reactions.
